(19) United States
(12) Patent Application Publication
(10) Pub. No.: US 2026/0328775 A1
(43) Pub. Date: Oct. 22, 2026

(54) METHOD FOR ISOLATING ANTIGEN MOLECULE

(71) Applicant: AICHI PREFECTURE, Nagoya-shi, Aichi (JP)

(72) Inventors: Ayumu TAGUCHI, Nagoya-shi, Aichi (JP); Yuichi ABE, Nagoya-shi, Aichi (JP); Yasuhide OKUMOTO, Nagoya-shi, Aichi (JP)

(73) Assignee: AICHI PREFECTURE, Nagoya-shi, Aichi (JP)

(21) Appl. No.: 19/114,676

(22) PCT Filed: Sep. 25, 2023
(86) PCT No.: PCT/JP2023/034650

§ 371 Date: Mar. 24, 2025

(30) Foreign Application Priority Data
Sep. 26, 2022 (JP) 2022-152089

(51) Int. Cl. G01N 33/68; G01N 1/40; G01N 30/72; G01N 33/575

(52) U.S. Cl. CPC G01N 33/6848; G01N 1/405; G01N 30/7233; G01N 33/575; G01N 2800/24

(57) ABSTRACT

Provided is a method of efficiently isolating and detecting an antigen molecule from an antigen-antibody complex in a biological sample. Provided is a method of isolating an antigen molecule from a liquid sample comprising an antigen-antibody complex, the method including: a binding step of binding the antibody molecule in the antigen-antibody complex to an antibody-binding protein by applying the liquid sample to a column on which the antigen-binding protein is immobilized; and an elution step of eluting an elution liquid comprising the antigen molecule by applying a buffer liquid to the column to dissociate the antigen molecule in the antigen-antibody complex from the antibody molecule, wherein the buffer liquid is applied to the column while the pH of the buffer liquid decreases in a continuous or stepwise manner in the elution step.

### TECHNICAL FIELD

The present invention relates to methods of isolating and detecting antigen molecules, and a method of assisting in differential diagnosis of a disease such as cancer and autoimmune disease.

### BACKGROUND ART

In a living organism, an antigen molecule, whether a self antigen or a non-self antigen, binds with an antibody to form an antigen-antibody complex. By analyzing an antigen-antibody complex present in the body, it may be possible to detect any changes in a living organism, such as a disease.

For example, in the humoral immune response against a tumor, an autoantibody that recognizes a cancer antigen appears in the bloodstream at an extremely early stage of the oncogenic process. It is known that an autoantibody recognizing a cancer antigen binds with a cancer antigen to form an antigen-antibody complex in a living organism, diffusing not only into tumor tissue but also into body fluid such as blood. Similarly, it is thought that an antibody against a pathogen-derived antigen for an infectious disease or an antibody against a self antigen for an autoimmune disease forms an antigen-antibody complex, diffusing into body fluid.

Therefore, analyzing an antigen-antibody complex in body fluid enables the efficient acquisition of information regarding the humoral immune response of the living organism. In particular, identifying the antigen molecule within the complex is expected to provide detailed information regarding cancer, an autoimmune disease, an infectious disease, or other diseases.

Non-patent Literature 1 describes a proteome analysis targeting a cancer antigen bound to an immunoglobulin in plasma. In this proteome analysis, antigen-antibody complexes recovered from plasma using beads bound to Protein A or G were subjected to mass spectrometry, resulting in identification of some of the antigen molecules. However, unfortunately, this method only identified a part of the relatively abundant antigen molecules, and trace amounts of antigen molecules cannot be identified due to being hindered by the large number of immunoglobulin molecules present in the sample.

Therefore, there is a need for the development of a method that overcomes the aforementioned problems in conventional techniques and enables the efficient isolation and detection of an antigen molecule from an antigen-antibody complex in a biological sample.

### CITATION LIST

### Non-Patent Literature

Non-patent Literature 1: Capello, M. et al., Nature Communications, 2019, 10:254.

### SUMMARY OF INVENTION

### Technical Problem

An object of the present invention is to provide methods of efficiently isolating and detecting an antigen molecule from an antigen-antibody complex in a biological sample.

### Solution to Problem

In conventional proteome analyses for antigen-antibody complexes, antigen-antibody complexes have been isolated from other biomolecules by using beads conjugated with antibody-binding proteins such as Protein A or G based on the binding between the antibody-binding proteins and immunoglobulins. During this isolation process, not only antigen-antibody complexes but also a large number of immunoglobulins that are not bound to antigen molecules are simultaneously collected by the beads. Therefore, during the elution step using acidic solvents or the like, a large number of immunoglobulins, along with the antigens dissociated from the immunoglobulins, have been eluted. The large number of contaminating immunoglobulins poses a significant obstacle to identifying the antigen molecules, and it is extremely difficult to selectively separate the antigen molecules from the antibody molecules.

To solve the above problems, the present inventors focused on the possibility of a difference between the binding strength of an antigen molecule with an antibody molecule and the binding strength of an antibody molecule with an antibody-binding molecule, conceiving that by utilizing this difference in binding strength, the antigen molecule could be selectively separated from the antigen-antibody complex (FIG. 1). Based on this idea, the present inventors allowed an antigen-antibody complex to be bound to a column with an antibody-binding molecule immobilized, and then eluted the antigen-antibody complex from the column using an eluent with a specific pH. This resulted in reduction of the amount of antibody molecules contaminated in the elution liquid, leading to the successful identification of more than twice the amount of an antigen molecule compared to conventional methods.

Furthermore, the present inventors performed pH gradient liquid chromatography to determine the optimal pH conditions for selectively separating an antigen molecule from an antibody molecule. This revealed highly advantageous elution conditions to allow dramatic reduction in the amount of antibody molecules contaminated and efficient detection of even trace amounts of antigen molecules, resulting in identification of six times or more the number of antigen molecules compared to conventional methods.

The present invention is based on the novel findings described above, and provides the following:
(1) A method of isolating an antigen molecule from a liquid sample comprising an antigen-antibody complex, the method comprising:
   a binding step of binding the antibody molecule in the antigen-antibody complex to an antibody-binding protein by applying the liquid sample to a column on which the antigen-binding protein is immobilized, and
   an elution step of eluting an elution liquid comprising the antigen molecule by applying a buffer liquid to the column to dissociate the antigen molecule in the antigen-antibody complex from the antibody molecule,
   wherein the buffer liquid is applied to the column while the pH of the buffer liquid decreases in a continuous or stepwise manner in the elution step.
(2) A method of detecting an antigen molecule from a liquid sample comprising an antigen-antibody complex, the method comprising:
   a binding step of binding the antibody molecule in the antigen-antibody complex to an antibody-binding protein by applying the liquid sample to a column on which the antigen-binding protein is immobilized,
   an elution step of eluting an elution liquid comprising the antigen molecule by applying a buffer liquid to the column to dissociate the antigen molecule in the antigen-antibody complex from the antibody molecule, and
   a detection step of detecting the antigen molecule in the elution liquid,
   wherein the buffer liquid is applied to the column while the pH of the buffer liquid decreases in a continuous or stepwise manner in the elution step.
(3) The method of (2), wherein the detection uses a mass spectrometry or immunological detection method.
(4) The method of any one of (1) to (3), wherein the antigen molecule is a self antigen.
(5) The method of any one of (1) to (3), wherein the elution step comprises decreasing the pH from pH 7.4 to pH 6.5, from pH 6.5 to pH 5.5, and/or from pH 5.5 to pH 4.5 in a continuous or stepwise manner.
(6) The method of (5), wherein the elution step comprises decreasing the pH from pH 7.4 to pH 4.5 in a continuous or stepwise manner.
(7) The method of any one of (1) to (3), wherein the antibody-binding protein is protein A, protein G or a fusion protein of protein A and protein G.
(8) The method of any one of (1) to (3), wherein the liquid sample is a body fluid, intraperitoneal lavage fluid, cell culture liquid, cell homogenization liquid, or tissue homogenization liquid.
(9) The method of (8), wherein the body fluid is selected from the group consisting of blood, serum, plasma, cerebrospinal fluid, urine, saliva, and ascitic fluid.
(10) The method of (8), wherein the body fluid or intraperitoneal lavage fluid is derived from a patient with a disease selected from the group consisting of cancer, autoimmune disease, collagen disease, infectious disease, inflammatory disease, and aging-related disease.
(11) A method of isolating an antigen molecule from a liquid sample comprising an antigen-antibody complex, the method comprising:
   a binding step of binding the antibody molecule in the antigen-antibody complex to an antibody-binding protein by contacting the liquid sample to a carrier on which the antigen-binding protein is immobilized.
   a dissociation step of dissociating the antigen molecule in the antigen-antibody complex from the antibody molecule by contacting a buffer liquid to the carrier, and
   a collection step of collecting the dissociated antigen molecule,
   wherein the pH of the buffer liquid is pH 4.5 to pH 7.4.
(12) The method of (11), wherein the pH of the buffer liquid is pH 6.5 to pH 7.4.
(13) The method of (11) or (12), wherein the antigen molecule is a self antigen.
(14) A method of assisting in differential diagnosis of cancer, comprising:
   a binding step of binding the antibody molecule in an antigen-antibody complex comprising a self-antigen molecule to an antibody-binding protein by applying a body fluid or intraperitoneal lavage fluid comprising the antigen-antibody complex derived from a subject and a control to a column on which the antigen-binding protein is immobilized,
   an elution step of eluting an elution liquid comprising the self-antigen molecule by applying a buffer liquid to the column to dissociate the antigen molecule in the antigen-antibody complex from the antibody molecule,
   a detection step of detecting the self-antigen molecule in the elution liquid, and
   a determination step of determining that the subject has cancer when the detection amount of a cancer antigen detected from the subject is greater than the detection amount of the cancer antigen detected from the control when the self-antigen molecules detected from the subject and the control are compared,
   wherein the buffer liquid is applied to the column while the pH of the buffer liquid decreases in a continuous or stepwise manner in the elution step.
(15) A method of assisting in differential diagnosis of an autoimmune disease, comprising:
   a binding step of binding the antibody molecule in an antigen-antibody complex comprising a self-antigen molecule to an antibody-binding protein by applying a body fluid or intraperitoneal lavage fluid comprising the antigen-antibody complex derived from a subject and a control to a column on which the antigen-binding protein is immobilized,
   an elution step of eluting an elution liquid comprising the self-antigen molecule by applying a buffer liquid to the column to dissociate the antigen molecule in the antigen-antibody complex from the antibody molecule,
   a detection step of detecting the self-antigen molecule in the elution liquid, and
   a determination step of determining that the subject has the autoimmune disease if the detection amount of a self antigen related to the autoimmune disease detected from the subject is greater than the detection amount of the self antigen detected from the control when the self-antigen molecules detected from the subject and the control are compared,
   wherein the buffer liquid is applied to the column while the pH of the buffer liquid decreases in a continuous or stepwise manner in the elution step.

The present specification encompasses the disclosure of Japan Patent Application No. 2022-152089, to which the present application claims the priority.

### Advantageous Effects of Invention

The present invention provides methods of efficiently isolating and detecting an antigen molecule from an antigen-antibody complex in a biological sample.

### BRIEF DESCRIPTION OF DRAWING

FIG. 1 schematically illustrates the difference between the binding strength of the antigen molecule with the antibody molecule (represented as “binding strength A” in the figure) and the binding strength of the antibody molecule with the antibody-binding protein (represented as “binding strength B” in the figure).

FIG. 2 shows the experimental procedure and the result of Example 2. FIG. 2A shows the experimental procedure of Example 2. The fractions 1 to 3 were collected by elution under weakly acidic conditions (pH 4.5), and then the fractions 4 to 5 were collected by elution under more acidic conditions (pH 2.8). FIG. 2B shows the result of electrophoresis on an acrylamide gel for the fractions 1 to 5. The strongly stained bands in fractions 4 to 5 correspond to the heavy/light chains of IgG antibodies. In the figure, “M” represents the lane of size marker.

FIG. 3 shows the changes in the pH of the eluents and the amounts of proteins in the elution liquids (absorbance at 280 nm) over time in the pH gradient liquid chromatography of FIG. 2.

FIG. 4 shows the results of the mass spectrometry analysis for the proteins in each elution fraction in FIG. 2. FIG. 4A shows the number of the identified antigen molecules by mass spectrometry. FIG. 4B shows the number of the identified antibody molecule by mass spectrometry.

FIG. 5 shows the amounts of the antigen molecules detected in each fraction for each type of the antigen molecule detected in Example 2 (number of identification by mass spectrometry).

FIG. 6 shows the amounts of the antibody molecules detected in each fraction for each type of the antibody molecule detected in Example 2 (number of identification by mass spectrometry).

FIG. 7 shows the results of detection of immunoglobulin (Ig) and antigen molecules in intraperitoneal lavage fluids. FIG. 7A shows the results of measurement by ELISA of the amounts of IgG in intraperitoneal lavage fluids derived from 16 pancreatic cancer surgery cases. The value for plasma samples shown on the left of the graph represents the plasma IgG theoretical value in healthy subjects. FIG. 7B shows the result obtained by performing pH gradient liquid chromatography on intraperitoneal lavage fluid (1 case) and a plasma sample (control) as test samples, followed by electrophoresis and staining of proteins for two fractions eluted at pH 4 and pH 2.

FIG. 8 shows the results of mass spectrometry analysis for immunoglobulin (Ig)-related proteins and non-Ig proteins (antigens) in intraperitoneal lavage fluids derived from 12 pancreatic cancer surgery cases comprising relapse-free cases (8 cases) and relapse cases (4 cases). The number of detections shown in the figure indicates the type of protein detected.

### DESCRIPTION OF EMBODIMENTS

### <Method of Isolating/Detecting Antigen Molecule>

In one aspect of the present invention, a method of isolating an antigen molecule from a liquid sample comprising an antigen-antibody complex (hereinafter, often abbreviated as “method of isolating an antigen molecule”) is provided. In an additional aspect of the present invention, a method of detecting an antigen molecule from a liquid sample comprising an antigen-antibody complex (hereinafter, often abbreviated as “method of detecting an antigen molecule”) is provided.

As used herein, the term “antigen-antibody complex” means a complex that is formed by the binding of an antigen molecule and an antibody molecule. The type of the antigen molecule and antibody molecule that constitute the antigen-antibody complex is not limited, and examples include antigen molecules and antibody molecules as described below.

As used herein, the term “antibody molecule” or “antibody” means a protein that exhibits immunoreactivity to an antigen. The antibody is not limited herein as long as it can form an antigen-antibody complex in a liquid sample such as body fluid. More specifically, the antibody is an immunoglobulin belonging to the immunoglobulin superfamily. Any class of immunoglobulins may be used, including IgG by way of example. Furthermore. IgG may be of any subclass, for example. IgG1, IgG2, IgG3, or IgG4.

As used herein, the term “antigen molecule” or “antigen” is any molecule to which an antibody binds. The antigen molecule is not particularly limited as long as an antibody can bind to it, and thus may be, for example, a protein or a peptide fragment, a sugar chain, or a lipid. The antigen may be derived from any origin, and may be a self antigen or a non-self antigen. When the antigen is a self antigen, the self antigen is not particularly limited, and may be, for example, a cancer antigen, or an antigen associated with an autoimmune disease. When the antigen is a non-self antigen, it may be, for example, a pathogen-derived antigen or an allergen. Examples of the pathogen-derived antigen include viral antigens, bacterial antigens, and fungal antigens. Examples of the allergen include inhalant allergens (e.g., pollens), food allergens (e.g., eggs, milk, wheat and buckwheat, and seafood such as shrimp), and contact allergens (e.g., feathers and metals). Antigens that can be isolated/detected by the method of the present invention correspond to antigens that can be dissociated from antibodies at a higher pH than antibody-binding proteins described below, and examples include antigens that bind to antibodies with a dissociation constant greater than the dissociation constant between the antibody and the antibody-binding protein described below.

As used herein, the term “cancer antigen” refers to an antigen with which a cancer cell can be distinguished from a normal cell. Cancer antigens, also called tumor antigens or tumor-associated antigens, correspond to biomolecules that are typically expressed in cancer cells in a specific manner and/or in an excessive amount. Cancer antigens are biomolecules such as proteins, sugar chains or lipids, and are not limited to full-length proteins consisting of wild-type amino acid sequences, and include peptide fragments and neoantigens. Specific examples of cancer antigens include, but not limited to, A33, BAGE, B-cell maturation antigen (BCMA), Bcl-2, β-catenin, CA19-9, CA125, carbonic anhydrase IX (CAIX), CCR4, CD5, CD19, CD20, CD21, CD22, CD24, CD33, CD37, CD45, CD123, CD133, CEA, CEACAM5, c-Met, CS-1, cyclin B1, DAGE, EBNA, EGFR, EpCAM, ephrin B2, estrogen receptor, FAP, folate-binding protein, GAGE, G250, disialoganglioside (GD2), GM2, gp75, gp100 (Pmel 17), ERBB2 (HER-2/neu), HPV E6, HPV E7, IGF1R, L1-CAM, LRP, MAGE, MART, mesothelin, MUCs (such as MUC1 and MUC2), MUM-1-B, myc, NYESO-1, p53, PRAME, progesterone receptor, PSA, PSCA, PSMA, ras, ROR1, survivin, SV40 T, tenascin, TNF-α, TSTA tyrosinase, VEGF, WT1, hTERT, and PAP.

As used herein, the term “self antigen” (autoantigen) means an antigen consisting of any biomolecule constituting one's own tissue or cells. The self antigen may be, for example, a protein, a sugar chain, or a lipid. It is noted that the simple reference to self antigen herein broadly means any autologous antigen molecule that binds, or can bind, to an antibody to form an antigen-antibody complex in the animal's body, and the self antigen is not limited to self antigens that cause autoimmune diseases.

As used herein, the term “antibody-binding protein” refers to a protein that can be used for antibody affinity purification, and is also called an antibody-adsorbing ligand. Specific examples of the antibody-binding protein include protein G, protein A, and modified or recombinant forms thereof. Protein G and protein A are antibody-binding proteins derived from Streptococcus and Staphylococcus aureus, respectively. Examples of the modified or recombinant forms include a fusion protein obtained by the fusion of protein A and protein G (hereinafter referred to as “protein A/G”). Depending on the antibody molecule of the target antigen-antibody complex in the method of the present invention, an antibody-binding protein that can specifically bind to the antibody molecule can be selected. The dissociation constant between the antibody and the antibody-binding protein is preferably 10⁻⁷ M or less. For example, they preferably have a high affinity of 10⁻⁸ M or less, and more preferably 10⁻⁹ M or less. The dissociation constants of protein A, protein G. and protein A/G range from 10⁻¹⁰ M to 10⁻¹¹ M. A dissociation constant in the above range allows for maintaining binding to the antibody under weakly acidic conditions. The antibody-binding protein is preferably protein G, protein A. or protein A/G.

As used herein, the term “column” or “separation column” means a vessel used to separate components in a sample in column chromatography, such as affinity chromatography. The column is packed with a solid support such as a resin, matrix, beads, or gel to which an antibody-binding protein is bound. The material of the solid support packed in the column is not limited, and may be, for example, agarose, sepharose, cellulose, glass, silica, polystyrene, collodion charcoal, polymethacrylate, zirconia, zeolite, vinyl polymer, polymethacrylate, polyacrylamide, polyacrylic acid, or dextran. Specific examples of “a column on which an antibody-binding protein are immobilized” herein include protein A columns, protein G columns, and protein A/G columns. The columns and the carriers described below are preferably equilibrated with balanced buffer liquid prior to application of or contact with a liquid sample.

The “carrier” used for the isolation of an antigen-antibody complex herein is not limited as long as an antibody-binding protein can be immobilized on the surface of the carrier and recovered by sedimentation, agglomeration, precipitation, centrifugation, filtration, magnetic force, or other recovery methods. For example, beads with an antibody-binding protein coated on their surface can be used. Beads are insoluble carriers in particle form, and their average particle size is, for example, 10 nm to 10 μm. The material of the beads is, but not particularly limited to, a polymer, a magnetic material, silica, agarose, or Sepharose. In particular, magnetic beads made of magnetic material are suitably used because they are easy to collect or purify.

As used herein, the term “elution” refers to a liquid that passes through a column and is discharged from the column. The method of elution is not limited, and may be, for example, gravity-based elution or elution based on the pressure from outside the column.

As used herein, the term “living organism” refers to a cell, tissue, an organ, or an individual. Without limitation, examples include an individual such as a human, livestock animal (such as cattle, horse, sheep, goat, pig, chicken, or ostrich), racehorse, companion animal (such as dog, cat, or rabbit), laboratory animal (such as mouse, rat, guinea pig, monkey, or marmoset), or wild or zoo animal (such as chimpanzee, wildcat, squirrel, eagle, or flamingo), or a cell, tissue, an organ, or a viscera derived therefrom.

As used herein, the term “liquid sample” refers to a sample that is applied with the method of isolating an antigen molecule or the method of detecting an antigen molecule of the present invention and can be applied to a column or contacted with a carrier. The liquid sample is not limited as long as it comprises an antigen-antibody complex, and corresponds to, for example, liquid derived from a living organism, such as body fluid, or a liquid sample prepared from any biological sample. Examples of preferred liquid samples include body fluid, intraperitoneal lavage fluid, cell culture liquid, cell homogenization liquid, and tissue homogenization liquid. A liquid sample may be derived from a healthy subject or patient with any disease. For example, a liquid sample may be derived from a patient with cancer, an autoimmune disease, a collagen disease, an infectious disease, an inflammatory disease, or an aging-related disease.

As used herein the term “biological sample” refers to any sample derived from a living organism Examples include tissue, a cell, and body fluid. Examples of the tissue include tissue homogenate, tissue section, and biopsy tissue. The tissue and cell correspond to tissue and cell suffering from or potentially suffering from a disease in a subject or corresponding tissue and a cell in a healthy subject, and may be, for example, tissue and cell derived from brain, liver, spleen, heart, pancreas (including pancreatic islets), lung, esophagus, kidney, ovary, stomach, large intestine, prostate, breast (including mammary gland), muscle, thyroid gland, and gingiva. A biological sample may be derived from a healthy subject or patient with any disease. For example, a biological sample may be derived from a patient with cancer, an autoimmune disease, a collagen disease, an infectious disease, an inflammatory disease, or an aging-related disease.

Examples of “body fluid” herein include cerebrospinal fluid, interstitial fluid, blood (including serum, plasma, and interstitial fluid), lymph, extracts of various tissue or cells, pleural fluid, sputum, lacrimal fluid, nasal discharge, saliva, urine, and ascitic fluid. Blood may be serum or plasma prepared from blood. For example, blood, serum, plasma, cerebrospinal fluid, urine, saliva, and ascitic fluid are preferred because they are relatively easy to collect. Body fluid may be derived from a healthy subject or patient with any disease. For example, body fluid may be derived from a patient with cancer, an autoimmune disease, a collagen disease, an infectious disease, an inflammatory disease, or an aging-related disease.

As used herein, the term “intraperitoneal lavage fluid” refers to a liquid that is introduced into the peritoneal cavity to wash the peritoneal cavity during surgery such as tumor resection and is collected after washing. The type of the lavage fluid is not particularly limited, but typically physiological saline is often used. An antigen molecule contained in the intraperitoneal lavage fluid can be a biomarker related to metastasis and prognosis of cancer.

As used herein, the type of “cancer” is not limited, and examples include adenocarcinoma, squamous cell carcinoma, small cell carcinoma, and large cell carcinoma. Specific examples of the type of the cancer include malignant melanoma, oral cancer, laryngeal cancer, pharyngeal cancer, thyroid cancer, lung cancer, breast cancer, esophageal cancer, gastric cancer, colorectal cancer (including colon cancer and rectal cancer), small intestine cancer, bladder cancer, prostate cancer, testis cancer, endometrial cancer, cervical cancer, endometrial cancer, ovarian cancer, gastric cancer, kidney cancer, liver cancer, pancreatic cancer, biliary tract cancer (including gallbladder cancer and bile duct cancer), brain tumor, head and neck cancer, mesothelioma, osteosarcoma, soft tissue sarcoma, glioma, childhood tumor including neuroblastoma, blood cancer, lymphoma, and myeloma. Examples of the blood cancer include leukemia (for example, B-cell leukemia, acute lymphoblastic leukemia, chronic lymphocytic leukemia), lymphoma (for example, non-Hodgkin's lymphoma), myeloma (for example, multiple myeloma).

As used herein, the term “autoimmune disease” refers to a disease in which the immune system triggers inappropriate responses against one's own cells, tissue, and/or organs, resulting in, for example, inflammation and injury. Examples of the autoimmune disease include lupus (e.g., systemic lupus erythematosus), celiac disease, acute disseminated encephalomyelitis, acute motor axonal neuropathy, Addison's disease, adiposis dolorosa (Dercum's disease), alopecia areata, ankylosing spondylitis, antiphospholipid syndrome, anti-synthetase syndrome, autoimmune pancreatitis, autoimmune thrombocytopenic purpura, autoimmune urticaria. Behcet's disease, Bickerstaff's encephalitis, bullous pemphigoid, celiac disease, chronic fatigue syndrome, chronic inflammatory demyelinating polyneuropathy, cicatricial pemphigoid, Crohn's disease, dermatomyositis, type 1 diabetes, endometriosis, fibromyalgia, gastritis, giant cell arteritis, Graves' disease, Graves ophthalmopathy, Guillain-Barre syndrome, Hashimoto's encephalopathy, Hashimoto's thyroiditis, juvenile arthritis, lichen planus, Lyme disease, multiple sclerosis, myasthenia gravis, myocarditis, neuromyelitis optica, Pediatric cute-onset neuropsychiatric syndrome, post-myocardial infarction syndrome, psoriasis, rheumatic fever, systemic lupus erythematosus, scleroderma, Sjogren's syndrome, transverse myelitis, ulcerative colitis, vasculitis, and vitiligo.

As used herein, the term “aging-related disease” means a disease that can develop with aging. Specific examples include collagen disease, diabetes, frailty, dementia. Alzheimer's disease, chronic kidney disease, and arteriosclerosis.

As used herein, the term “subject” refers to a living organism to be applied with the method of isolating an antigen molecule, the method of detecting an antigen molecule, and the method of assisting in differential diagnosis of a disease of the present invention. For example, a human (in this case, specifically referred to as “subject”), livestock animal (such as cattle, horse, sheep, goat, and pig), racehorse, companion animal (such as dog, cat, and rabbit), laboratory animal (such as mouse, rat, guinea pig, monkey, and marmoset), and wild or zoo animal (such as chimpanzee, wildcat, and squirrel) are included. For example, a living organism such as a human or mammal that has or is at risk of developing a disease such as cancer or an autoimmune disease is included.

As used herein, the term “healthy subject” refers to an individual who does not suffer from a specific disease, preferably an individual who does not suffer from any disease. However, in this specification, healthy tissue is also included in the broad sense of healthy subjects. Therefore, a subject will be referred to as a healthy subject not only w % ben the subject is in a healthy state at the individual level but also at the tissue level, for example, as a normal portion of tissue taken from a patient.

As used herein, the term “control” corresponds to “healthy subject,” corresponding to an individual that does not suffer from a specific disease (e.g., cancer or autoimmune disease), preferably an individual that does not suffer from any disease. A control may be a single individual or a plurality of individuals.

As used herein, the term “detection” can be used interchangeably with test, measurement, determination, or determination support.

As used herein, the term “separation” refers only to an increase in the proportion of the component of interest compared to the other components, and is not limited to the complete separation of a particular component from the other components. For example, “separating an antigen molecule from an antibody molecule” means an increase in the proportion of the antigen molecule contained in the sample compared to the antibody molecule. This includes, for instance, preparing a sample in which the proportion of the antigen molecule to the antibody molecule has increased compared to the pre-separation sample or obtaining such a fraction.

The method of isolating an antigen molecule and the method of detecting an antigen molecule of the present invention involve different steps depending on whether the antigen-antibody complex is bound to a column or to a carrier. In the following description, when distinguishing between the method involving binding to a column and the method involving binding to a carrier, the isolation method where the antigen-antibody complex is bound to a column is referred to as a “method of isolating an antigen molecule using a column”, while the isolation method where the antigen-antibody complex is bound to a carrier is referred to as a “method of isolating an antigen molecule using a carrier” for the distinction. Similarly, the detection method where the antigen-antibody complex is bound to a column is referred to as a “method of detecting an antigen molecule using a column”, while the detection method where the antigen-antibody complex is bound to a carrier is referred to as “method of isolating an antigen molecule using a carrier” for the distinction.

### (Method of Isolating/Detecting an Antigen Molecule Using a Column)

The method of isolating an antigen molecule using a column of the present invention comprises a binding step and an elution step, as essential steps. The method of detecting an antigen molecule using a column of the present invention comprises a binding step, an elution step, and a detection step, as essential steps. The configurations of the steps will be described below.

In the method of isolating an antigen molecule using a column and the method of detecting an antigen molecule using a column of the present invention, the “binding step” is a step of binding the antibody molecule in the antigen-antibody complex to an antibody-binding protein by applying the liquid sample to a column on which the antigen-binding protein is immobilized. In this step, the term “applying” to a column can also be translated as “loading” or “adding.” In this step, a liquid sample is applied to a column, and as a result, the liquid sample can flow through the column (eluted).

In this step, the column is preferably equilibrated in advance to the pH at which the antigen-antibody complex can bind to the antibody-binding protein. Similarly, the liquid sample is also preferably prepared and equilibrated to the pH at which the antigen-antibody complex can bind to the antibody-binding protein by, for example, mixing the liquid sample with a loading buffer liquid before the application to the column.

The pH of the liquid sample applied to the column and the column in the binding step is preferably at or higher than the pH of the eluting buffer liquid used in the elution step as described below at the start of elution. The pH is, for example, pH 5.4 or higher, pH 6.4 or higher, pH 6.8 or higher, pH 7.4 or higher, or pH 8.0 or higher, and preferably is a pH within the neutral range (for example, pH 6.4 to pH 8.6, pH 6.8 to pH 8.0, or pH 7.4). A component or a buffering agent contained in the liquid sample for pH adjustment is not particularly limited, and includes, for example, phosphate, citric acid, succinic acid, acetic acid, ammonium hydroxide, Tris, glycine, phosphoric acid, Good buffer, boric acid, sodium carbonate, and HEPES.

In the method of isolating an antigen molecule using a column and the method of detecting an antigen molecule using a column of the present invention, the “elution step” is a step of eluting an elution liquid comprising the antigen molecule by applying a buffer liquid to the column to dissociate the antigen molecule in the antigen-antibody complex from the antibody molecule. This step is characterized in that the buffer liquid applied to the column for elution (hereinafter referred to as “eluting buffer liquid”) is applied to the column while the pH of the buffer liquid is lowered in a continuous or stepwise manner.

As used herein, the phrase “applying the buffer liquid to the column while lowering the pH of the buffer liquid in a continuous manner” specifically means that pH gradient is used as the eluting buffer liquid. Methods for preparing pH gradient are well known in the art. For example, pH gradient can be prepared by mixing two or more buffer liquids with different pH (for example, pH 4.0 and pH 7.4/pH 2.7 and pH 7.4) while continuously changing the mixing ratio.

As used herein, the phrase “applying the buffer liquid to the column while lowering the pH of the buffer liquid in a stepwise manner” refers to application of eluting buffer liquids having one or more pH different from the binding step described above to a column. The pH interval at which the pH is changed in a stepwise manner is not limited.

In one embodiment, the pH range over which the pH is lowered in a continuous or stepwise manner in the elution step is within the range from pH 7.4 to pH 4.5. The pH that is the starting point in the elution step is pH 7.4 or less, and may be, for example, pH 7.3, pH 7.2, pH 7.1, pH 7.0, pH 6.9, pH 6.8, pH 6.7, pH 6.6, pH 6.5, pH 6.4, pH 6.3, pH 6.2, pH 6.1, pH 6.0, pH 5.9, pH 5.8, pH 5.7, pH 5.6, pH 5.5, pH 5.4, pH 5.3, pH 5.2, pH 5.1, pH 5.0, pH 4.9, or pH 4.8 or less. The pH that is the end point in the elution step is pH 4.5 or more, and may be, for example, pH 4.6, pH 4.7, pH 4.8, pH 4.9, pH 5.0, pH 5.1. pH 5.2, pH 5.3, pH 5.4, pH 5.5, pH 5.6, pH 5.7, pH 5.8, pH 5.9, pH 6.0, pH 6.1, pH 6.2, pH 6.3, pH 6.4, pH 6.5, pH 6.6, pH 6.7, pH 6.8, pH 6.9, or pH 7.0 or more. Exemplary pH ranges include the range from pH 7.4 to pH 6.5, from pH 6.5 to pH 5.5, and/or from pH 5.5 to pH 4.5. Preferably, the pH can be lowered from pH 7.4 to pH 4.5 in a continuous or stepwise manner. By using a pH in the above range, the antigen molecule can be dissociated from the antibody molecule with little or substantially no dissociation of the antibody molecule from the carrier.

Examples of the eluting buffer liquid include an eluent comprising a buffering component such as Tris, Good buffer, an acetate ion, a citrate ion, a succinate ion, a phosphate ion, a formate ion, a propionate ion, glycine, gamma-aminobutyric acid, or lactic acid. In addition, a surfactant (Tween 20 or Triton-X100), urea, guanidine, or an amino acid such as arginine or lysine can be added.

The rate of elution is not limited as long as the rate allows the eluting buffer liquid to slowly pass through the column, and may be, for example, 0.1 mL/min to 5 mL/min.

The amount of the eluting buffer liquid used in the elution step is usually 1 to 10 times the column volume, preferably 3 to 5 times.

The elution step provides an elution liquid eluted from the column. The elution liquid comprises an antigen molecule dissociated from the antibody molecule, and comprises a different antigen molecule based on the pH at which it is eluted, and thus is preferably obtained in multiple fractions. For fractionation, a fraction collector may be used.

In the method of detecting an antigen molecule using a column of the present invention, the “detection step” is a step of detecting the antigen molecule in the elution liquid obtained in the elution step described above.

The method for detecting an antigen molecule in the detection step may be any known protein detection method, such as a mass spectrometry or immunological detection method. The mass spectrometry method is not limited, and for example, the Data dependent analysis (DDA), Data independent analysis (DIA). Parallel reaction monitoring (PRM), or Selection reaction monitoring (SRM) method can be used as appropriate by those skilled in the art.

As used herein, the “immunological detection method” is a method of detecting an antigen molecule of interest using an antibody or an antibody fragment that specifically binds to the antigen molecule. Examples of the immunological detection method include enzyme immunoassays (including ELISA and EIA), Western blotting, radioimmunoassay (RIA), immunoprecipitation, and flow cytometry.

Both the mass spectrometry and immunological detection methods are known techniques in the art. Therefore, specific measurements may be performed according to a known method. For example, methods described in Green, M. R and Sambrook, J., 2012, Molecular Cloning: A Laboratory Manual Fourth Ed., Cold Spring Harbor Laboratory Press, Cold Spring Harbor, New York can be used as a reference.

The method of isolating an antigen molecule using a column and the method of detecting an antigen molecule using a column of the present invention can also comprise a preparation step and/or a washing step as an optional step in addition to the essential steps described above.

The “preparation step” is a step of preparing a liquid sample from a sample collected from a living organism. For example, a body fluid sample can be prepared to the pH at which the antigen-antibody complex can bind to the antibody-binding protein by mixing the sample with a loading buffer liquid. For a tissue sample or a cell sample, a liquid sample can be prepared by homogenization or solubilization. By carrying out this step before the binding step, a liquid sample that can be applied to the column can be prepared.

The “washing step” is a step of applying a washing buffer liquid to the column after the binding step and before the elution step to elute, from the column, components that are not bound to the antibody-binding protein, for example, contaminants other than the antigen-antibody complex or the antibody. The washing buffer liquid used in the washing step is a solution used to remove residual contaminants on the column. The washing buffer liquid preferably does not allow the antigen-antibody complex to be eluted from the column. For example, a buffer liquid having the same pH as the loading buffer liquid can also be used. The amount of the washing buffer liquid used in the washing step is usually 1 to 20 times the column volume, preferably 3 to 5 times. The number of washings is, for example, one or more times, and can be, for example, one, two, three, or four times.

### (Method of Isolating/Detecting an Antigen Molecule Using a Carrier)

The method of isolating an antigen molecule using a carrier of the present invention comprises a binding step, a dissociation step, and a collection step, as essential steps. The method of detecting an antigen molecule using a carrier of the present invention comprises a binding step, a dissociation step, a collection step, and a detection step, as essential steps. The configurations of the steps will be described below.

In the method of isolating an antigen molecule using a carrier and the method of detecting an antigen molecule using a carrier of the present invention, the “binding step” is a step of binding the antibody molecule in the antigen-antibody complex to an antibody-binding protein by contacting the liquid sample to a carrier on which the antigen-binding protein is immobilized.

The carrier used in the binding step is preferably equilibrated in advance to the pH at which the antigen-antibody complex can bind to the antibody-binding protein. Similarly, the liquid sample is also preferably prepared and equilibrated to the pH at which the antigen-antibody complex can bind to the antibody-binding protein by, for example, mixing the liquid sample with a buffer liquid before the contact with the carrier.

The pH at which the liquid sample is contacted with the carrier in the binding step is preferably higher than that of the dissociating buffer liquid used in the dissociation step as described below. The pH is, for example, pH 5.4 or higher, pH 6.4 or higher, pH 6.8 or higher, pH 7.4 or higher, or pH 8.0 or higher, and preferably is a pH within the neutral range (for example, pH 6.4 to pH 8.6, pH 6.8 to pH 8.0, or pH 7.4).

The method for contacting the liquid sample with the carrier in the binding step is not limited. For example, the liquid sample can be mixed with the carrier and, if necessary, allowed to stand or be shaken.

The carrier after the binding step can be collected by sedimentation, aggregation, precipitation, filtration, centrifugation, magnetic force, or the like depending on the type of the carrier. The carrier can also be washed before the dissociation step as described below to allow components that are not bound to the antibody-binding protein, for example, other components than the antigen-antibody complex or the antibody, to be removed from the carrier. The washing buffer liquid used in the washing step preferably does not allow the antigen-antibody complex bound to the carrier to be dissociated. For example, a buffer liquid having the same pH as the buffer liquid used in the contact as described above can also be used. The number of washings is, for example, one or more times, and can be, for example, one, two, three, or four times.

In the method of isolating an antigen molecule using a carrier and the method of detecting an antigen molecule using a carrier of the present invention, the “dissociation step” is a step of dissociating the antigen molecule in the antigen-antibody complex from the antibody molecule by contacting a buffer liquid to the carrier after the binding step.

The pH of the buffer liquid used in the dissociation step (hereinafter referred to as “dissociating buffer liquid”) is pH 4.5 to pH 7.4. Exemplary pH includes pH 5.0 to pH 7.0, pH 5.4 to pH 6.5, or pH 5.8 to pH 6.2. In addition. pH 6.5 to pH 7.4, pH 5.5 to pH 6.4, and pH 4.5 to pH 5.4 are also included. By using a pH in the above range, the antigen molecule can be dissociated from the antibody molecule with little or substantially no dissociation of the antibody molecule from the carrier.

Examples of the dissociating buffer liquid include buffer liquid comprising a buffering component such as Tris, Good buffer, an acetate ion, a citrate ion, a succinate ion, a phosphate ion, a formate ion, a propionate ion, glycine, gamma-aminobutyric acid, or lactic acid. In addition, a surfactant (Tween 20 or Triton-X100), urea, guanidine, or an amino acid such as arginine or lysine can be added.

The dissociation step allows for dissociation of the antigen molecule from the antibody molecule bound to the carrier.

In the method of detecting an antigen molecule using a carrier of the present invention, the “collection step” is a step of collecting the dissociated antigen molecule after the dissociation step. The collection method is preferably a method that allows for separation of the antigen molecule dissociated into the dissociating buffer liquid from the carrier. Preferably, the carrier is removed by, for example, sedimentation, aggregation, precipitation, filtration, centrifugation, or magnetic force depending on the type of the carrier, and then the dissociating buffer liquid comprising the antigen molecule is collected. The antigen molecule collected by the collection step can also be further purified, for example, by electrophoresis, such as SDS-PAGE with acrylamide gel, followed by cutting out the area corresponding to the size of interest.

The method of isolating an antigen molecule using a carrier and the method of detecting an antigen molecule using a carrier of the present invention can also comprise a preparation step and/or detection step as an optional step. These steps can be performed according to the method described above in “(Method of isolating/detecting an antigen molecule using a column)”, and therefore, the explanation is omitted here.

### <Method of Assisting in Differential Diagnosis of Cancer/Autoimmune Disease>

In one aspect of the present invention, a method of assisting in differential diagnosis of cancer (hereinafter, often abbreviated as “cancer differentiation method”) is provided. In a further aspect of the present invention, a method of assisting in differential diagnosis of an autoimmune disease (hereinafter, often abbreviated as “autoimmune disease differentiation method”) is also provided.

The cancer differentiation method of the present invention comprises a binding step, an elution step, a detection step, and a determination step as essential steps, and a preparation step and/or a washing step as optional steps. The autoimmune disease differentiation method of the present invention comprises a binding step, an elution step, a detection step, and a determination step as essential steps, and a preparation step and/or a washing step as optional steps.

The binding step in the cancer differentiation method and the autoimmune disease differentiation method of the present invention (hereinafter, the two methods referred to together as “differentiation method of the present invention”) is performed according to the binding step described above in the “(Method of isolating/detecting an antigen molecule using a column)” except that body fluid or intraperitoneal lavage fluid derived from a subject and a control, comprising an antigen-antibody complex comprising a self-antigen molecule is used as a liquid sample, and thus its detailed description is omitted here. The elution step and the detection step in the differentiation method of the present invention are performed according to the elution step and the detection step described above in the “(Method of isolating/detecting an antigen molecule using a column)” except that a self-antigen molecule is eluted/detected as an antigen molecule, and thus their detailed descriptions are omitted here. The preparation step and the washing step as optional steps in the differentiation method of the present invention are also performed according to the preparation step and the washing step described in the “(Method of isolating/detecting an antigen molecule using a column)”, and thus their detailed descriptions are omitted here.

In the cancer differentiation method of the present invention, the determination step is a step of determining that a subject has cancer when the detection amount of a cancer antigen detected from the subject is greater than the detection amount of the cancer antigen detected from a control when a self-antigen molecule detected from the subject and the control are compared.

In the autoimmune disease differentiation method of the present invention, the determination step is a step of determining that a subject has an autoimmune disease if the detection amount of a self antigen related to the autoimmune disease detected from the subject is greater than the detection amount of the self antigen detected from the control when a self-antigen molecule detected from the subject and the control are compared.

As used herein, the term “self antigen related to an autoimmune disease” means an antigen that is targeted by the immune system when the immune system induces an inappropriate response against its own cells, tissue, and/or organs in an autoimmune disease described above. Many self antigens related to autoimmune diseases are known. Specific examples include, but not limited to, self antigens related to idiopathic thrombocytopenic purpura (e.g., glycoprotein IIb/Illa, glycoprotein Ib/IX), self antigens related to Basedow's disease (e.g., anti-thyroid-stimulating hormone receptor, TSHR), self antigens related to systemic lupus erythematosus (SLE) (e.g., non-histone nucleoprotein antigens and other nuclear antigens (Sm), PCNA), self antigens related to multiple sclerosis (e.g., proteolipid protein PLP-1, myelin basic protein MBP, α-B-crystallin), self antigens related to type 1 diabetes (e.g., insulin, glutamic acid decarboxylase 2, tyrosine phosphatase IA2/IA-2p, Glima38, P52, GLUT-2), self antigens related to Guillain-Barre syndrome (e.g., peripheral myelin protein 1), self antigens related to myasthenia gravis (e.g., cholinergic receptor alpha chain), self antigens related to autoimmune thyroiditis (e.g., thyroglobulin), self antigens related to rheumatoid arthritis (e.g., collagen, fibrin, GP-39, hnRNP, MMP-1), self antigens related to autoimmune hepatitis (e.g., cytochrome p450), self antigens related to Graves' disease (e.g., thyroid-stimulating hormone receptor), and self antigens related to celiac disease (e.g., transglutaninase).

The method of comparing self-antigen molecules derived from a subject and control in the determination step in the differentiation method of the present invention is not limited. An exemplary method includes determining a cutoff value for the amount of a self-antigen molecule detected in a subject based on the amount of the self-antigen molecule detected in control, and comparing the results based on this cutoff value. In other words, a predetermined value is defined as a cutoff value, and if the measured value is greater than or equal to that value, it can be determined that the amount of a self-antigen molecule from a subject is greater than the amount of the self-antigen molecule from control.

The cutoff value is a boundary value for comparing the amounts of a self-antigen molecule in a subject and control. The cutoff value can usually be calculated based on the sensitivity and specificity determined based on the disease incidence and ROC curve (receiver operating characteristic curve). The method of setting cutoff values is not limited.

For example, the amount of a self-antigen molecule in a healthy subject not affected by cancer or an autoimmune disease, or the average amount of a self-antigen molecule in the healthy subject group is used as a cutoff value, and when the amount of the self-antigen molecule in a subject is greater than the cutoff value, it can be determined that the amount of the self-antigen molecule in the subject is greater than the amount of the self-antigen molecule in the control.

Alternatively, 1.5 times or more, 2 times or more, 3 times or more, 4 times or more, 5 times or more, or 6 times or more the amount of a self-antigen molecule in a healthy subject not affected by cancer or autoimmune diseases, or the average amount of a self-antigen molecule in the healthy subject group can be used as a cutoff value is used as a cutoff value, and when the amount of the self-antigen molecule in a subject is greater than the cutoff value, it can be determined that the amount of the self-antigen molecule in the subject is greater than the amount of self-antigen molecule in the control.

The measurements obtained from the control group can also be classified by percentiles, and the percentile values used for the classification can be used as the cutoff value. For example, the 95th percentile of the amount of self-antigen molecules obtained from the control is used as a cutoff value, and if the amount of a self-antigen molecule in a subject is 95th percentile or more, it can be determined that the amount of the self-antigen molecule in the subject is greater than the amount of the self-antigen molecule in the control.

The amount of self-antigen molecules in the control healthy subject does not necessarily have to be measured each time unlike the amount of a self-antigen molecule in a subject. For example, if the amount of sample used for the measurement, the measurement method, and the measurement conditions are kept constant, the amount of self-antigen molecules in the control healthy subject that was previously measured can be reused.

According to the differentiation method of the present invention, detection of a self-antigen molecule in body fluid collected from a subject allows quick and easy differential diagnosis of cancer or an autoimmune disease.

According to the differentiation method of the present invention, a therapeutic strategy (e.g., type, dosage, and interval of administration of anticancer agents or drugs for treating autoimmune diseases) can be determined, and/or at least one of drug therapy and radiation therapy can be performed based on the results of the differential diagnosis.

The differentiation method of the present invention may also be combined with another method (e.g., radiography; ultrasound (echo) examination; endoscopy; mammography; palpation; internal examination; rectal examination; CT scan; MRI scan; PET-CT scan or other imaging tests, a blood test; a pathology test such as cytology or histology; a tumor marker test; and/or genetic diagnosis) to improve the accuracy of differential diagnosis.

### EXAMPLES

### Example 1: Isolation of Antigen Molecule Using Weakly Acidic Eluent

### (Purpose)

Normal body fluid samples derived from living organisms are rich in antibody molecules. Separation of antibody molecules from other biomolecules can be performed using antibody-binding proteins such as Protein A. However, antigen molecules in antigen-antibody complexes in which the antigen molecules and antibody molecules are bound cannot be separated from the antibody molecules based on the same method.

In a sample comprising an abundance of specific proteins such as antibodies, it is not easy to detect other proteins, and it is especially difficult to detect trace proteins, even using mass spectrometry.

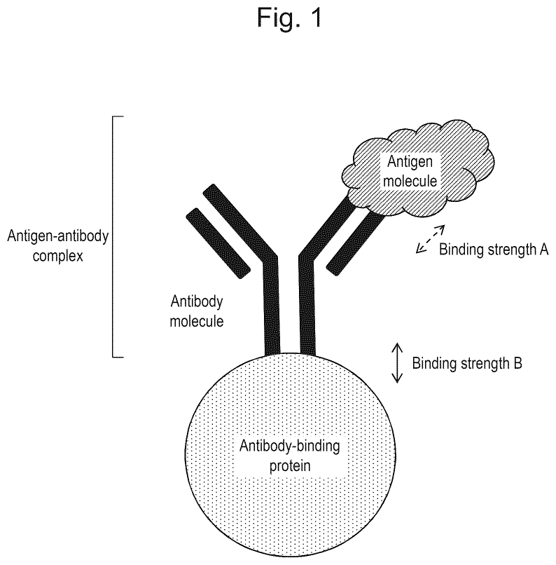

Therefore, in this Example, based on the possibility of a difference between the binding strength of antigen molecules with antibody molecules and the binding strength of antibody molecules with protein A (FIG. 1), the effectiveness of anew method for selectively separating antigen molecules from antigen-antibody complexes bound to protein A is tested. Specifically, antigen-antibody complexes in human plasma are bound to protein A immobilized on a spin column. Thereafter, the possibility of separation of the antigen molecules and the antibody molecules by a two-step elution using two eluents with different pH is investigated.

### (Method)

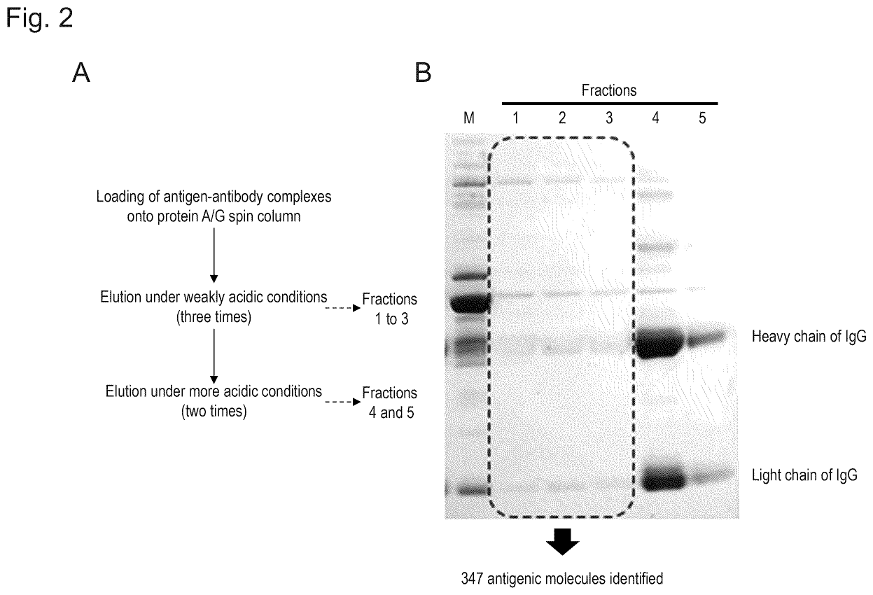

The following procedure was used for this Example. The procedure of this Example is shown schematically in FIG. 2A.

### (1) Antigen Separation Using Spin Column

Plasma was separated from blood collected from patients with colorectal cancer by centrifugation. Forty microliters of the plasma were mixed with a buffer liquid to prepare a liquid sample at pH 8. The liquid sample was loaded onto a spin column (Thermo Fisher Scientific #89954), and then centrifuged at 5,000×g for 60 seconds to allow antigen-antibody complexes in the plasma to bind to Protein A/G (fusion protein with the antibody binding sites of Protein A and Protein G fused together) immobilized on the spin column. Binding buffer at pH 8 was applied to the spin column and stirred at room temperature for 30 minutes for equilibration. Centrifugation was then performed at 5,000×g for 60 seconds, followed by two washes with Binding buffer.

Next, an eluent at pH 4.5 (composition: 100 mM glycine) was applied, followed by centrifugation at 5,000×g for 60 seconds to obtain an elution liquid of fraction 1. The same elution process was repeated two more times to obtain elution liquids of fractions 2 and 3 (FIG. 2A).

Thereafter, an eluent at pH 2.8 (Thermo Fisher Scientific #21004) was loaded, followed by centrifugation at 5,000×g for 60 seconds to obtain an elution liquid of fraction 4. The same elution process was repeated to obtain an elution liquid of fractions 5 (FIG. 2A).

### (2) Detection of Protein

Equal volumes of ethanol were then added to the fractions 1 to 5 (500 μL each) for protein precipitation, followed by solubilization in 1% SDS buffer. Electrophoresis was performed on a 4%-12% Bis-Tris polyacrylamide gel at 200 V, 400 mA, followed by protein staining with SimplyBlue™ SafeStain (Thermo Fisher Scientific #LC6060). The gels, after electrophoresis of the fractions 1 to 5, were digested with trypsin to obtain peptide samples for mass spectrometry, from which proteins were detected using a mass spectrometer Q Exactive (Thermo Fisher Scientific #IQLAAEGAAPFALGMAZR).

### (Results)

The results are shown in FIG. 2B.

The fractions 1 to 3 eluted with the eluent at pH 4 showed little or no detection of heavy/light chain bands of IgG antibodies. On the other hand, the fractions 4 and 5 eluted with the eluent at pH 2 showed strong detection of heavy/light chain bands of IgG antibodies.

Then, mass spectrometry of the proteins recovered from the gels after electrophoresis of the fractions 1 to 3 and fractions 4 and 5 resulted in the identification of 347 proteins. In the method described in previous literature (Capello, M. et al., Nature Communications, 2019, 10:254.), more than half of the identified proteins were composed of contaminated antibodies, but in this Example, over 70% of the identified proteins were antigen proteins.

This result is significantly higher than the detection of 154 proteins, which was previously reported based on conventional methods and exceeded the common protein identification criteria (FDR<0.01) (Capello, M. et al., Nature Communications, 2019, 10:254), demonstrating that the method of the present invention allows for the detection of antigen molecules with very high efficiency.

### Example 2: High-Precision Isolation of Antigen Molecule Based on pH Gradient Liquid Chromatography

### (Purpose)

After antigen-antibody complexes in which antigen molecules and antibody molecules are bound are bound to protein A. pH gradient liquid chromatography is applied to determine the optimal pH conditions for selectively separating only antigen molecules. Further, it is investigated whether antigen molecules can be separated based on the difference in the pH conditions under which antigen molecules can be dissociated from antibody molecules.

### (Method)

### (1) pH Gradient Liquid Chromatography

Plasma was separated from blood collected from mice (strain name: C57BL/6, sex: female) by centrifugation. Forty microliters of the plasma were mixed with a buffer liquid to prepare a liquid sample at pH 7.4. The liquid sample was loaded onto SkillPak™ column (Tosoh Corporation, #45201) on which Protein A was immobilized. Subsequently, elution was performed using eluents with pH gradients from pH 7.4 to pH 4.0. The pH gradient eluate was prepared by mixing 25 mM sodium citrate buffer liquids adjusted to pH 7.4 and pH 2.7. Specifically, the ratio of pH 2.7 buffer liquid mixed to pH 7.4 buffer liquid was continuously increased from 0% to 73%, while a total of 20 mL of eluent was passed through the column, followed by 8 mL of the 73% mixture. The flow rate was set at 1.0 mL/min. Finally, an eluent at pH 2.7 was passed through the column to elute the remaining proteins. The elution liquids were collected as different fractions for each 1.0 mL. Proteins contained in the elution liquids were monitored over time by absorbance at 220 nm (detection wavelength for peptide bonds)/280 nm (detection wavelength for aromatic amino acids).

### (2) Identification and Analysis of Protein

The protein concentration in each fraction obtained was determined using a DC protein assay kit (BIO-RAD, #5000111). Peptide samples for mass spectrometry were then prepared from the protein solutions. Liquid samples comprising peptide fragments were prepared by performing treatments such as desalting, solubilization, and concentration or drying, as needed, followed by re-dissolution in a solvent for mass spectrometry. A system incorporating a field asymmetric ion mobility spectrometry interface (Thermo Fisher Scientific #FMS02-10001) in a high-resolution tandem mass spectrometer coupled with high-performance liquid chromatography (Thermo Fisher Scientific #ULTIM3000RSLCNANO, BRE725535) was used to measure the signal intensity of the full mass spectrum.

### (Results)

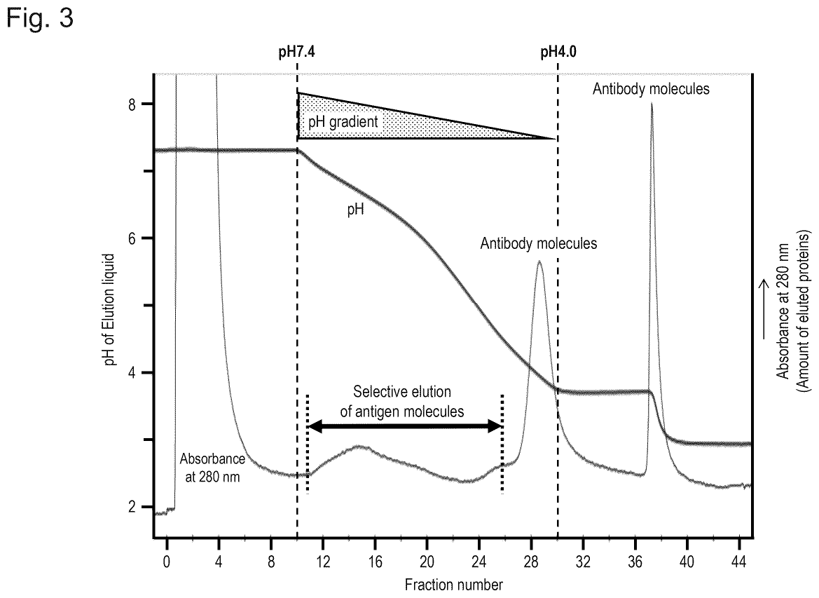

The changes in the pH of the eluents and the amounts of proteins in the elution liquids (absorbance at 280 nm) over time in the pH gradient liquid chromatography are shown in FIG. 3.

The amount of protein in the elution liquid showed a small peak between pH 7.4 and pH 4.5, followed by a large peak corresponding to the elution of antibody molecules until pH 4.0. These results suggest that antigen molecules were selectively eluted during the change in the pH of the eluate from 7.4 to 4.5, while antibody molecules remained bound to the column and were barely eluted.

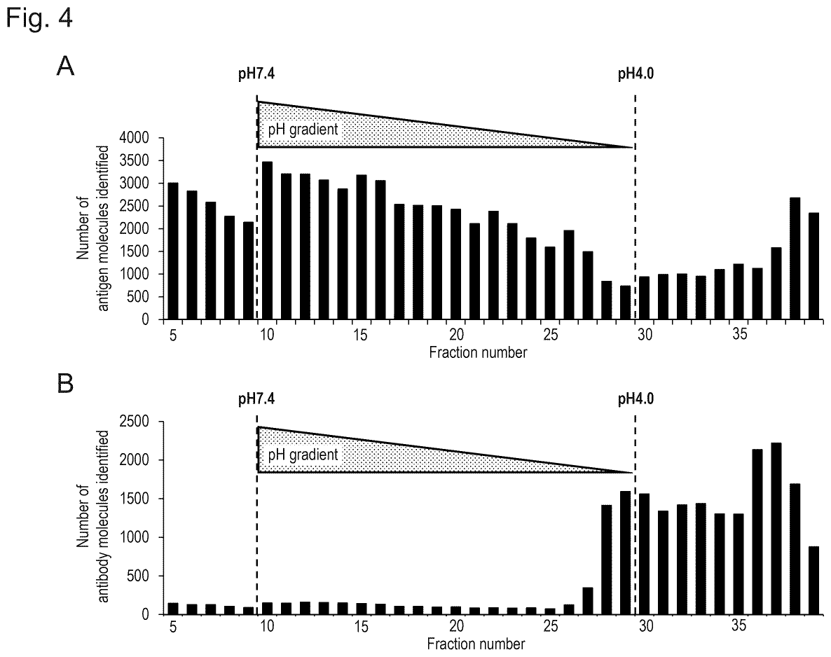

The results of the mass spectrometry analysis for the proteins in each elution fraction are shown in FIG. 4. FIGS. 4A and 4B show the number of the identified antigen and antibody molecules, respectively. Note that these results do not represent the types of the antigen molecules, since the number of identifications corresponds to the total number of proteins detected, regardless of the types of the proteins. Antigen molecules were selectively eluted over the fractions from 10 to 26 in the pH gradient (FIG. 4A). In contrast, few antibody molecules were eluted in fractions before the start of the pH gradient (fractions 5 to 9) and in fractions from the beginning to the end of the pH gradient (fractions 10 to 26), while large amounts were eluted from the fraction 27 onward (FIG. 4B).

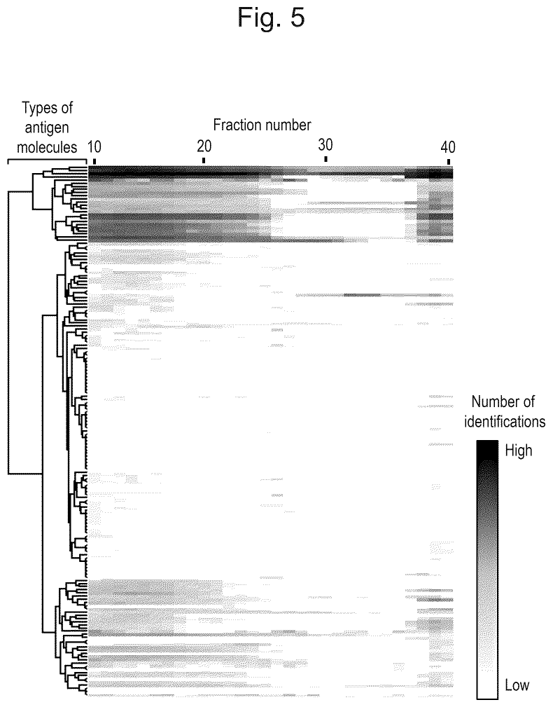

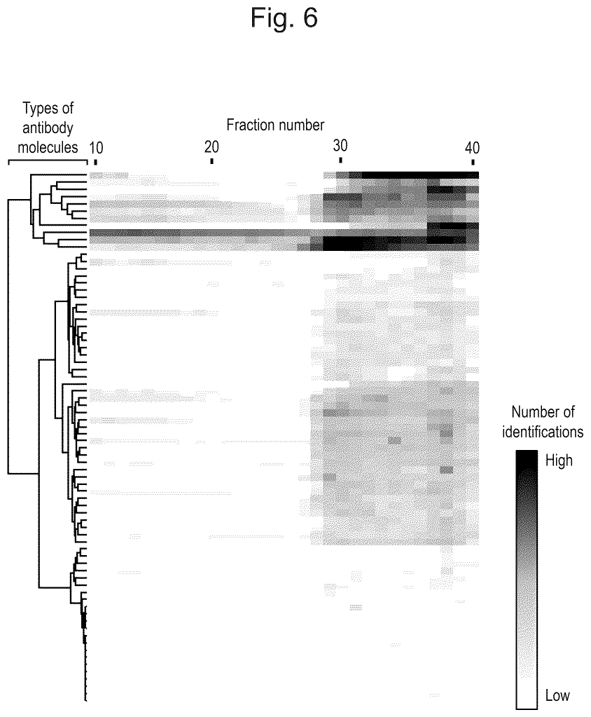

FIGS. 5 and 6 show the amounts of antigen and antibody molecules detected in the fractions, respectively, categorized by the type of the antigen or antibody molecule. These results indicate that antigen molecules were selectively eluted during the change in the pH of the eluate from 7.4 to 4.5 (in the range from fraction 10 to fraction 26), while most of the antibody molecules remained bound to the column and were not eluted. This finding demonstrates that this pH range is highly advantageous for detecting antigenic proteins present in extremely small amounts in blood, such as self antigens and cancer-specific antigens.

### Example 3: Isolation of Antigen Molecule from Plasma Obtained from Patient with Colorectal Cancer

### (Purpose)

Using plasma obtained and prepared from patients with colorectal cancer, antigen molecules contained in antigen-antibody complexes are isolated and identified.

### (Methods and Results)

In this Example, a total of eight plasma samples from patients with colorectal cancer were tested. Specifically, this Example used plasma collected at the Aichi Cancer Center Hospital with patients' consent, and was conducted with the approval of the Ethics Committee of Aichi Cancer Center. The pH gradient liquid chromatography and protein identification and analysis were performed according to the method described in Example 2.

As a result, an average of 988 antigen molecules were detected from a total of the eight plasma samples derived from patients with colorectal cancer, and 1227 antigen molecules were detected in the eight plasma samples combined.

The detection results of Examples 1-3 are shown in Table 1 below, with the results in a previous report based on conventional methods (Capello, M. et al., Nature Communications, 2019, 10:254.) used as a comparison.

TABLE 1
Comparison of the results from Examples
1-3 and conventional method

| | Sample type | Identified antigen molecules |
|---|---|---|
| Example 1 | plasma from a patient | 347 |
| | with colorectal cancer | |
| Example 2 | plasma from a mouse | 377 |
| Example 3 | plasma from a patient | 930-1015 |
| | with colorectal cancer | (n = 8; 988 on average) |
| Previous | plasma from a patient | 109-215 |
| report (*) | with pancreatic cancer | (n = 10; 154 on average) |

*Capello, M. et al., Nature Communications, 2019, 10: 254.

As shown in Table 1 above, by using the antigen separation method using an eluate prepared at a specific weakly acidic pH as described in Example 1 herein, twice or more antigen molecules were successfully identified compared to the conventional method. Furthermore, by using the antigen separation methods using the pH gradient described in Examples 2 and 3 herein achieved the identification of two to six times or more antigen molecules compared to the conventional method.

### Example 4: Isolation/Detection of Antigen Molecule from Intraperitoneal Lavage Fluid

### (Purpose)

Using intraperitoneal lavage fluid obtained from patients with pancreatic cancer, an antigen molecule contained in an antigen-antibody complex is isolated and identified.

### (Methods and Results)

### (1) Intraperitoneal Lavage Fluid

In this Example, intraperitoneal lavage fluids derived from 16 pancreatic cancer surgery cases were tested. This Example used intraperitoneal lavage fluid collected at the Aichi Cancer Center Hospital with patients' consent, and was conducted with the approval of the Ethics Committee of Aichi Cancer Center.

### (2) Detection of IgG in Intraperitoneal Lavage Fluid

The amounts of immunoglobulin G (IgG) in the intraperitoneal lavage fluids derived from 16 pancreatic cancer surgery cases were measured by ELISA.

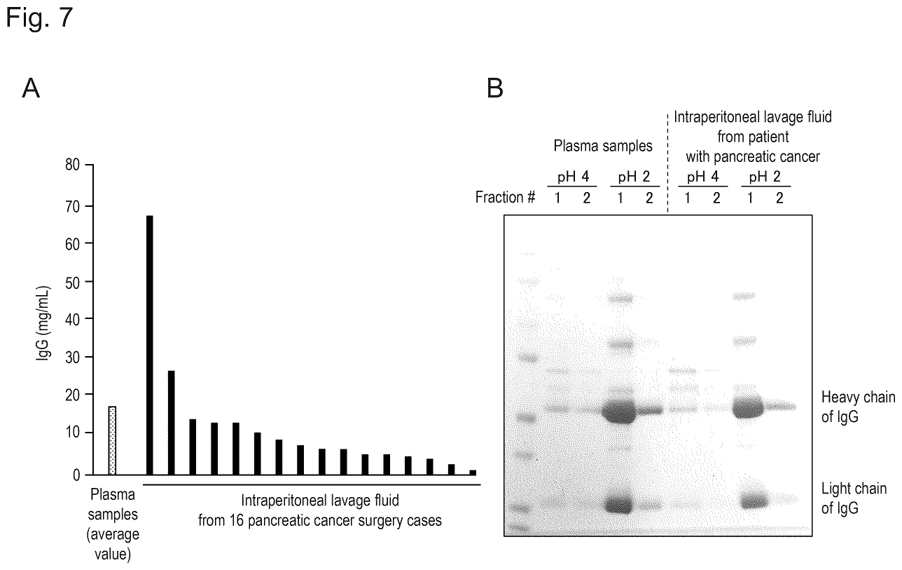

The results are shown in FIG. 7A. In FIG. 7A, the plasma IgG theoretical value in healthy subjects as a control is shown in the graph. It was revealed that the intraperitoneal lavage fluid from patients with pancreatic cancer comprised a large amount of IgG, similar to that in plasma.

### (3) Isolation of an Antigen Molecule Based on pH Gradient Liquid Chromatography

pH gradient liquid chromatography was performed on intraperitoneal lavage fluids derived from 12 pancreatic cancer surgery cases comprising relapse-free cases (8 cases) and relapse cases (4 cases) according to the method described in Example 2.

An equal volume of ethanol was first added to elution liquids at pH 4 and pH 2 for protein precipitation, followed by solubilization in 1% SDS buffer. Electrophoresis was performed on a 4%-12% Bis-Tris polyacrylamide gel at 200 V, 400 mA, followed by protein staining with SimplyBlue™ SafeStain (Thermo Fisher Scientific #LC6060). As a control, plasma samples derived from healthy subjects were similarly subjected to pH gradient liquid chromatography and electrophoresis/staining, and compared with the results of intraperitoneal lavage fluid.

The results of analyzing elution liquids at pH 4 and pH 2 (two fractions each) for an intraperitoneal lavage fluid sample (one case) and a plasma sample (control) are shown in FIG. 7B. In the intraperitoneal lavage fluid from pancreatic cancer, as in the plasma samples, the elution liquid at pH 4 resulted in little or no detection of heavy/light chain bands of IgG antibodies, while the elution liquid at pH 2 resulted in strong detection of heavy/light chain bands of IgG antibodies. These results revealed that antigens/antibodies can be separated and purified using intraperitoneal lavage fluid from pancreatic cancer.

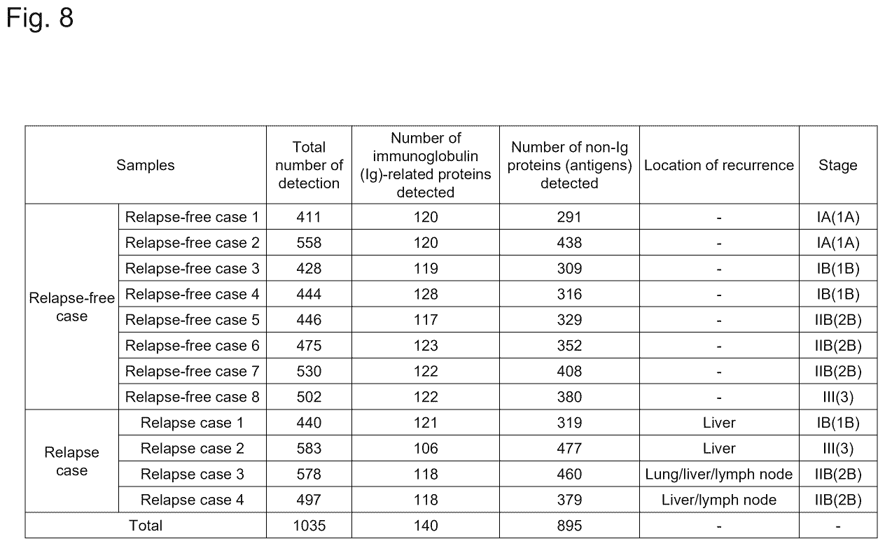

Next, the results of mass spectrometry analysis of the elution liquid from pH gradient liquid chromatography according to the method described in “(2) Identification and analysis of protein” in Example 2 are shown in FIG. 8. From each of the pancreatic cancer relapse-free cases (8 cases) and pancreatic cancer relapse cases (4 cases), 400 to 600 proteins were detected (FIG. 8, “Total number of detected proteins”). Among them, antigens not related to immunoglobulin (Ig) (FIG. 8, “Number of detected non-Ig proteins (antigens)”) excluding proteins related to immunoglobulin (Ig) (FIG. 8, “Number of detected immunoglobulin (Ig)-related protein”) were in the range of 200 to 500 types.

These results demonstrated that antigen molecules in antigen-antibody complexes can be detected in pancreatic cancer intraperitoneal lavage fluid by a method using pH gradient liquid chromatography.

All publications, patents, and patent applications cited herein are incorporated herein by reference in their entirety.

1. A method of isolating an antigen molecule from a liquid sample comprising an antigen-antibody complex, the method comprising:
a binding step of binding the antibody molecule in the antigen-antibody complex to an antibody-binding protein by applying the liquid sample to a column on which the antigen-binding protein is immobilized, and
an elution step of eluting an elution liquid comprising the antigen molecule by applying a buffer liquid to the column to dissociate the antigen molecule in the antigen-antibody complex from the antibody molecule,
wherein the buffer liquid is applied to the column while the pH of the buffer liquid decreases in a continuous or stepwise manner in the elution step.

2. A method of detecting an antigen molecule from a liquid sample comprising an antigen-antibody complex, the method comprising:
a binding step of binding the antibody molecule in the antigen-antibody complex to an antibody-binding protein by applying the liquid sample to a column on which the antigen-binding protein is immobilized,
an elution step of eluting an elution liquid comprising the antigen molecule by applying a buffer liquid to the column to dissociate the antigen molecule in the antigen-antibody complex from the antibody molecule, and
a detection step of detecting the antigen molecule in the elution liquid,
wherein the buffer liquid is applied to the column while the pH of the buffer liquid decreases in a continuous or stepwise manner in the elution step.

3. The method of claim 2, wherein the detection uses a mass spectrometry or immunological detection method.

4. The method of claim 1, wherein the antigen molecule is a self antigen.

5. The method of claim 1, wherein the elution step comprises decreasing pH from pH 7.4 to pH 6.5, from pH 6.5 to pH 5.5, and/or from pH 5.5 to pH 4.5 in a continuous or stepwise manner.

6. The method of claim 5, wherein the elution step comprises decreasing pH from pH 7.4 to pH 4.5 in a continuous or stepwise manner.

7. The method of claim 1, wherein the antibody-binding protein is protein A, protein G or a fusion protein of protein A and protein G.

8. The method of claim 1, wherein the liquid sample is a body fluid, intraperitoneal lavage fluid, cell culture liquid, cell homogenization liquid, or tissue homogenization liquid.

9. The method of claim 8, wherein the body fluid is selected from the group consisting of blood, serum, plasma, cerebrospinal fluid, urine, saliva, and ascitic fluid.

10. The method of claim 8, wherein the body fluid or intraperitoneal lavage fluid is derived from a patient of a disease selected from the group consisting of cancer, autoimmune disease, collagen disease, infectious disease, inflammatory disease, and aging-related disease.

11. A method of isolating an antigen molecule from a liquid sample comprising an antigen-antibody complex, the method comprising:
a binding step of binding the antibody molecule in the antigen-antibody complex to an antibody-binding protein by contacting the liquid sample to a carrier on which the antigen-binding protein is immobilized,
a dissociation step of dissociating the antigen molecule in the antigen-antibody complex from the antibody molecule by contacting a buffer liquid to the carrier, and
a collection step of collecting the dissociated antigen molecule,
wherein the pH of the buffer liquid is pH 4.5 to pH 7.4.

12. The method of claim 11, wherein the pH of the buffer liquid is pH 6.5 to pH 7.4.

13. The method of claim 11, wherein the antigen molecule is a self antigen.

14. A method of diagnosing cancer, comprising:
a binding step of binding the antibody molecule in an antigen-antibody complex comprising a self-antigen molecule to an antibody-binding protein by applying a body fluid or intraperitoneal lavage fluid comprising the antigen-antibody complex derived from a subject and a control to a column on which the antigen-binding protein is immobilized,
an elution step of eluting an elution liquid comprising the self-antigen molecule by applying a buffer liquid to the column to dissociate the antigen molecule in the antigen-antibody complex from the antibody molecule,
a detection step of detecting the self-antigen molecule in the elution liquid, and
a determination step of determining that the subject has cancer when the detection amount of a cancer antigen detected from the subject is greater than the detection amount of the cancer antigen detected from the control when the self-antigen molecules detected from the subject and the control are compared,
wherein the buffer liquid is applied to the column while the pH of the buffer liquid decreases in a continuous or stepwise manner in the elution step.

15. A method of diagnosing an autoimmune disease, comprising:
a binding step of binding the antibody molecule in an antigen-antibody complex comprising a self-antigen molecule to an antibody-binding protein by applying a body fluid or intraperitoneal lavage fluid derived from a subject and a control to a column on which the antigen-binding protein is immobilized,
an elution step of eluting an elution liquid comprising the self-antigen molecule by applying a buffer liquid to the column to dissociate the self-antigen molecule in the antigen-antibody complex from the antibody molecule,
a detection step of detecting the self-antigen molecule in the elution liquid, and
a determination step of determining that the subject has the autoimmune disease if the detection amount of a self antigen related to the autoimmune disease detected from the subject is greater than the detection amount of the self antigen detected from the control when the self-antigen molecules detected from the subject and the control are compared,
wherein the buffer liquid is applied to the column while the pH of the buffer liquid decreases in a continuous or stepwise manner in the elution step.
